(19) United States
(12) Patent Application Publication
(10) Pub. No.: US 2008/0223377 A1
(43) Pub. Date: Sep. 18, 2008

(54) BREATHING ENHANCEMENT DEVICE AND METHOD OF ASSEMBLING AND USING THE SAME

(76) Inventor: Leon Kussick, Naples, FL (US)

Correspondence Address: SHERIDAN ROSS PC, 1560 BROADWAY, SUITE 1200, DENVER, CO, 80202, US

(21) Appl. No.: 12/015,344

(22) Filed: Jan. 16, 2008

Related U.S. Application Data
(60) Provisional application No. 60/885,200, filed on Jan. 16, 2007.

(51) Int. Cl. A61F 5/56; A61F 5/37

(52) U.S. Cl. 128/848; 128/869

(57) ABSTRACT

An appliance for breathing enhancement that includes a support member and securing mechanism for holding a user's head in a position where the user's mouth is closed and the user's head is tilted to maintain an open airway. The breathing enhancer reduces or eliminates snoring. In at least one embodiment of the invention, the teeth are maintained in a normal closed bite position (1-5 mm open) while the lips are touching or almost touching to eliminate or substantially eliminate open mouth breathing.

### CROSS-REFERENCE TO RELATED APPLICATIONS

The present application claims the benefit of U.S. Provisional Patent Application Ser. No. 60/885,200 filed Jan. 16, 2007, the entire content of which is considered to be part of the disclosure of the present application, and hereby incorporated by reference in its entirety as if fully set forth herein.

### FIELD

The present invention relates generally to a breathing enhancement device, and more particularly, to a device that can be worn by a user to improve breathing and/or reduce the tendency to snore.

### BACKGROUND

Many individuals have difficulty breathing when sleeping, and such breathing difficulties can be caused by a person's position when sleeping. More particularly, a person's head and/or neck position can influence the person's ability to breathe properly when sleeping. The inability to breathe adequately when sleeping can lead to health problems. In addition, improper posture when sleeping can also influence a person's tendency to snore. As many people know, snoring can be irritating to other individuals who may be subjected to the person's snoring.

An existing device known as the Continuous Positive Airway Pressure (CPAP) Mask tries to improve the breathing process by providing pressurized air through a mask that covers the patient's nose and mouth. Many patient's find the CPAP Mask to be uncomfortable for a variety of reasons. For example, many people who have tried a CPAP Mask find it to be claustrophobic and very noisy due to the fan.

In addition to the CPAP Mask, various surgery techniques also try to improve the breathing process with varying degrees of success. The techniques may include treatment to remove soft palette and uvula, ablate (cut away) the back of the tongue and destroy tongue muscles (scar tissue shrinks and reduces the tongue size), and cut the mandible (ramus) and bring the jaw forward. However, many people find that surgery does not improve their breathing process.

Thus, there is a need to provide an apparatus and/or treatment method of assisting people who have difficulty breathing. In addition, there is a need to provide an apparatus and/or treatment method of assisting people who have difficulty breathing while they sleep. In addition, there is a need to provide an apparatus and/or treatment method of assisting people who snore.

### SUMMARY

Various embodiments of the present invention address the shortcomings of the prior art. It is to be understood that the present invention includes a variety of different versions or embodiments, and this Summary is not meant to be limiting or all inclusive. This Summary provides some general descriptions of some of the embodiments, but may also include some more specific descriptions of certain embodiments.

Embodiments of the present invention include an appliance to enhance the breathing process, including the breathing process while the user of the appliance is sleeping. The appliance is a breathing enhancer that comprises a support member or chin pillow that is adjustably secured around the individual's neck with a strap. The support member or chin pillow serves to maintain the jaw of the wearer of the appliance in a closed position while creating a normal bite. The normal bite is maintained without protruding the mandible or opening the mouth.

In use, the support member of the breathing enhancer is worn under the chin and lies firmly against the hyoid bone (or Adam's apple). When correctly sized, embodiments of the present invention do not adversely affect breathing or blood circulation, but rather, the breathing enhancer will reduce snoring by tilting the head backward, which then pulls the tongue forward to open the airway. By doing so, the posterior airway is opened with air flowing through the nose.

Various embodiments of the present invention are set forth in the attached figures and in the detailed description of the invention as provided herein and as embodied by the claims. It should be understood, however, that this Summary does not contain all of the aspects and embodiments of the present invention, is not meant to be limiting or restrictive in any manner, and that the invention as disclosed herein is and will be understood by those of ordinary skill in the art to encompass obvious improvements and modifications thereto.

Additional advantages of the present invention will become readily apparent from the following discussion, particularly when taken together with the accompanying drawings.

### BRIEF DESCRIPTION OF THE DRAWINGS

FIG. 1 is a front-side perspective view of an embodiment of the present invention;

FIG. 2 is a front elevation view of the device shown in FIG. 1;

FIG. 3 is a right side elevation view of the device shown in FIG. 1;

FIG. 4 is a left side elevation view of the device shown in FIG. 1;

FIG. 5 is a rear elevation view of the device shown in FIG. 1;

FIG. 6 is a top plan view of the device shown in FIG. 1;

FIG. 7 is a bottom plan view of the device shown in FIG. 1;

FIG. 8 is a front perspective view of the device shown in FIG. 1;

FIGS. 9 and 10 are bottom perspective views of the device shown in FIG. 1;

FIGS. 11 and 12 are top perspective views of the device shown in FIG. 1;

FIG. 13 is a top perspective view of an embodiment of the present invention; and

FIG. 14 is a side perspective view of an embodiment of the present invention being worn by a user.

The drawings are not necessarily drawn to scale.

### DETAILED DESCRIPTION

In accordance with embodiments of the present invention, a device is provided for improving a person's breathing. The device can be worn by the person when sleeping. Embodiments of the present invention include a strap and a support member. When worn, the strap maintains the position of the support member below the person's chin, thereby holding the person's head in a position for improving the flow of air when the person breathes.

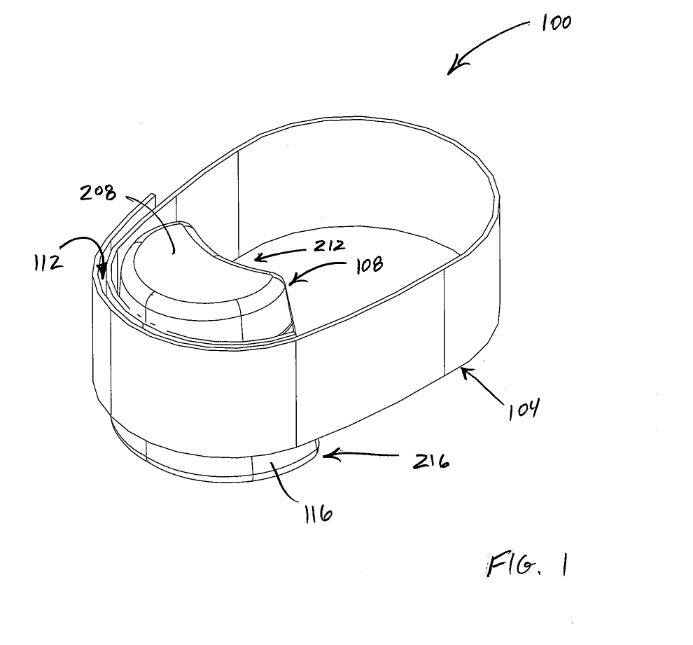

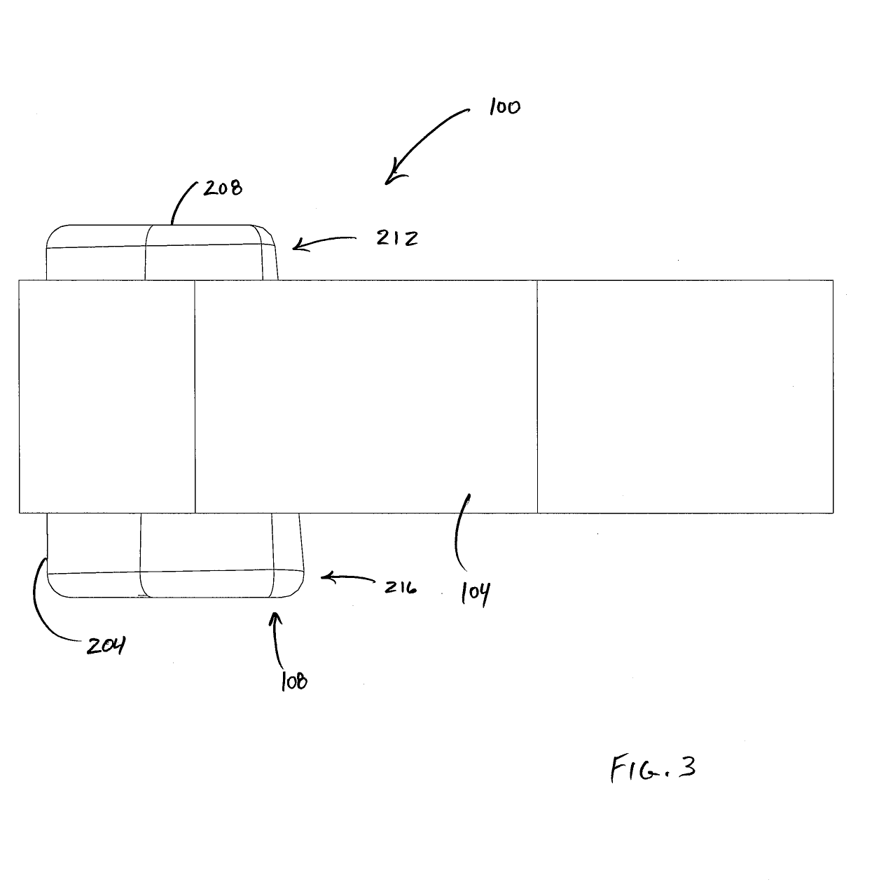

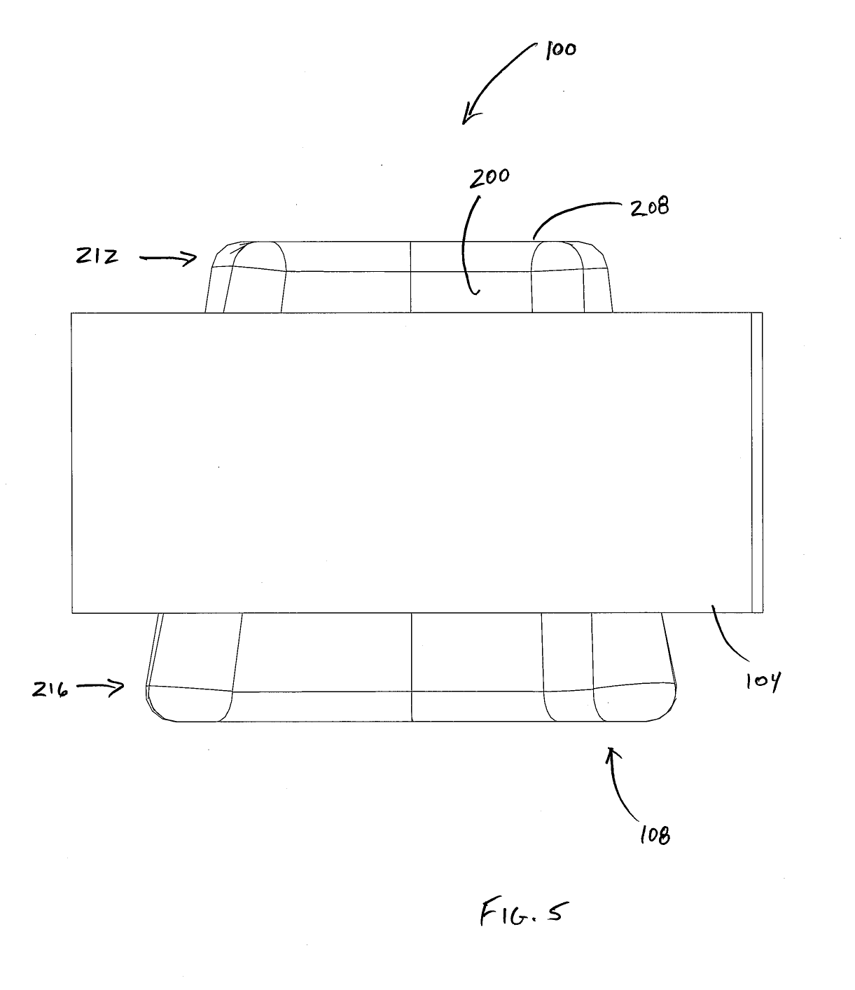

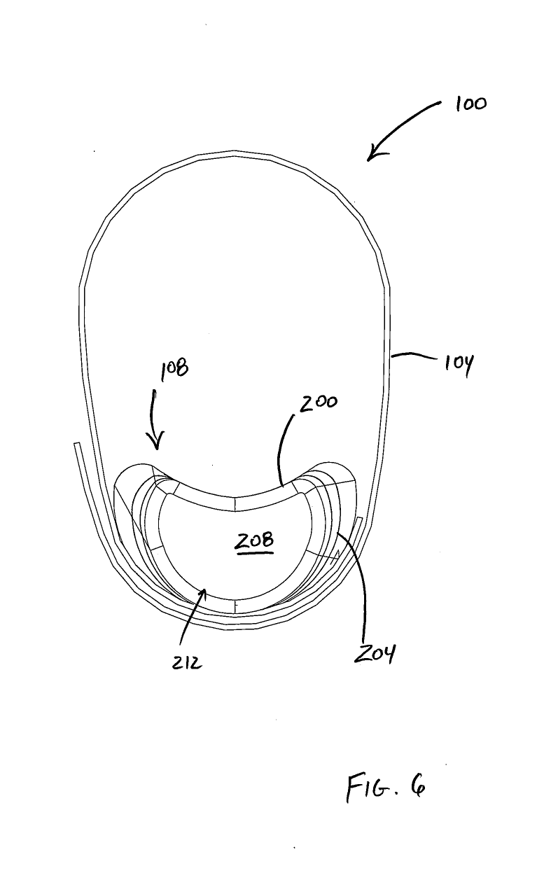

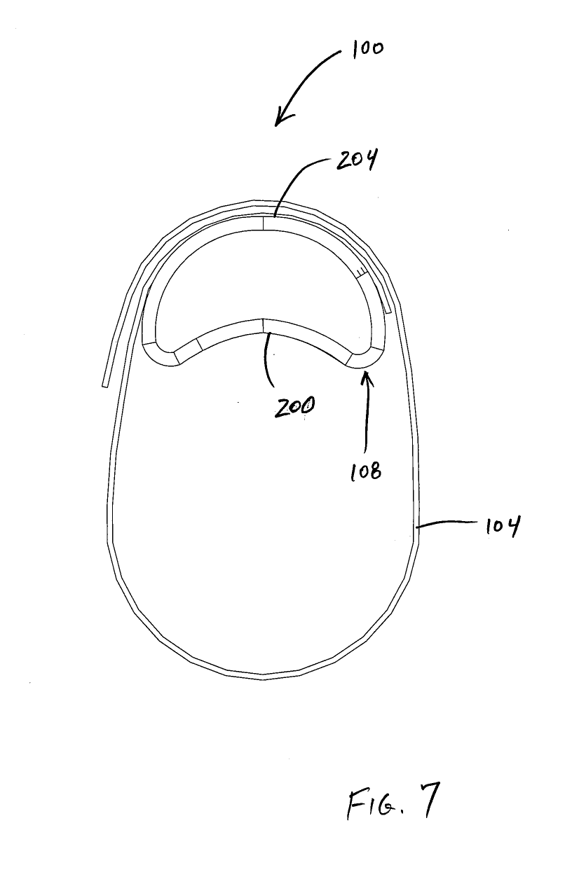

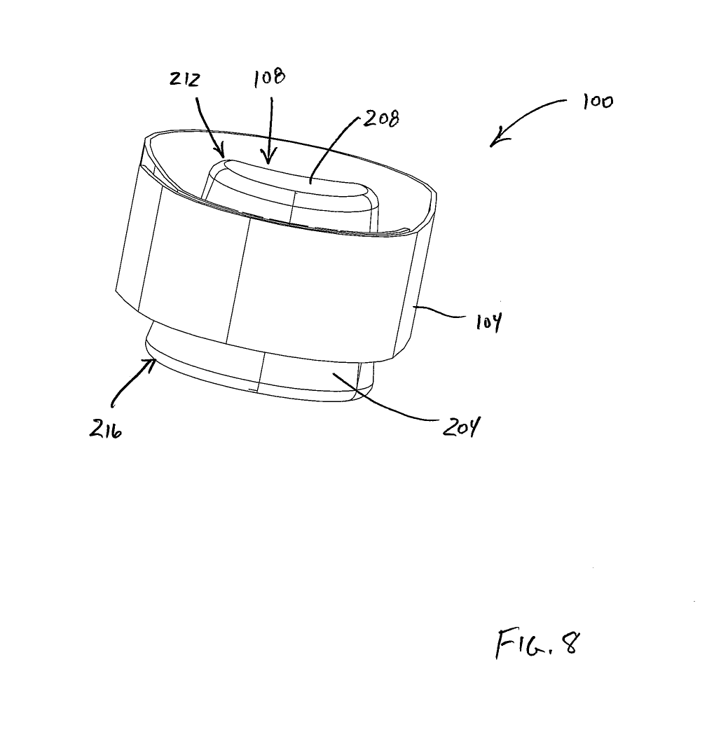

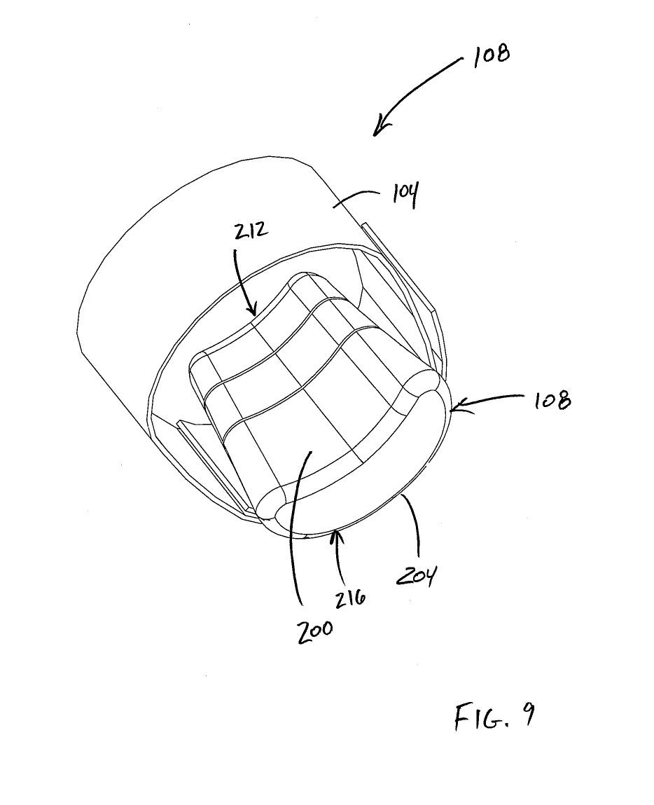

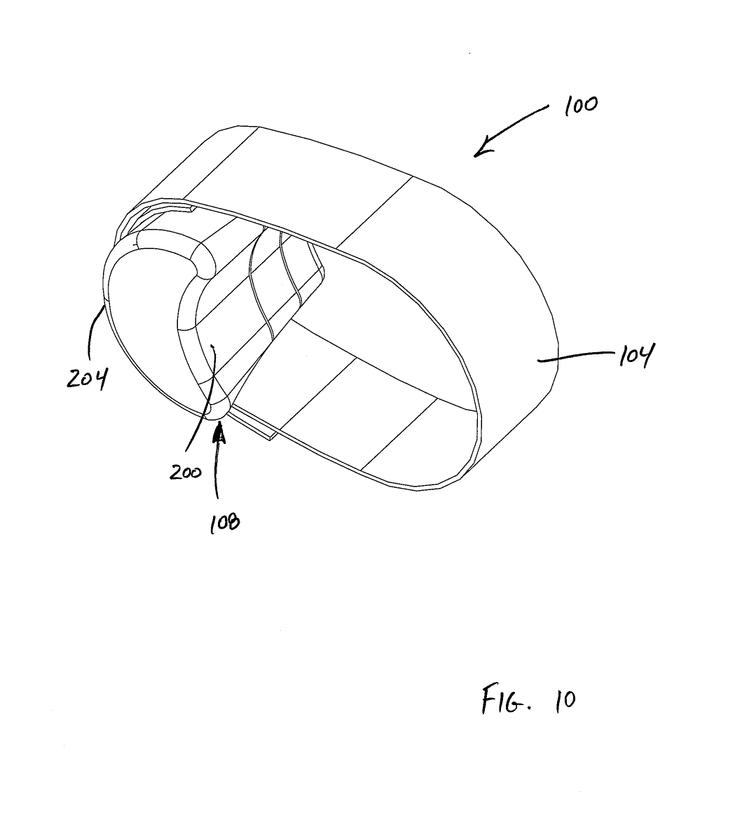

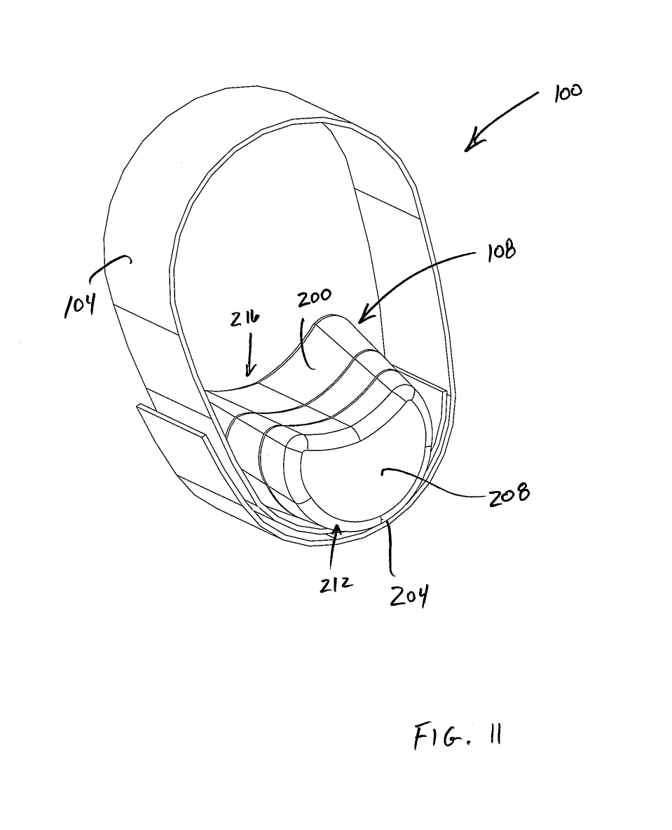

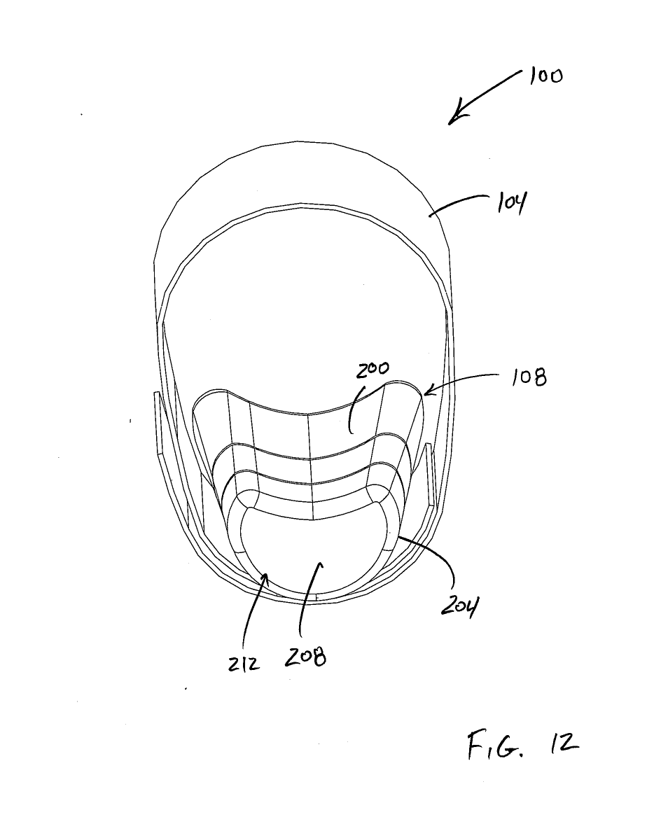

Referring now to FIGS. 1-12, an embodiment of the present invention is shown. The breathing enhancer 100 includes a strap 104 and a support member 108. In accordance with embodiments of the present invention, the strap 104 comprises a relatively soft material that is adjustable and can be fastened around the neck of the wearer of the breathing enhancer 100 to hold the support member 108 in place. Accordingly, the strap 104 preferably includes an adjustable securing mechanism 112, such as a hook-and-loop fastener (e.g., VELCRO®). However, those skilled in the art will appreciate that the strap 104 may be secured by another type of securing mechanism 112, such as by one or more of a buckle, button, clip, snap, etc. The strap 104 may be non-stretchable, or alternatively, the strap 104 may comprise a stretchable material, such as strands or bands of an elastic or rubber/rubber-like material.

The support member 108 preferably comprises a relatively soft member, such as a pillow type of structure, in the form of a shaped body 116 that is formed for positioning below the chin of the wearer so that the support member 108 maintains the wearer's head in a supported position. In at least one embodiment of the invention, the support member 108 is detachably attached to the strap 104, such as by a hook-and-loop fastener. Alternatively, the strap 104 may pass through slits or a sleeve in the support member 108, and/or the strap 104 may pass through a covering associated with the support member 108. However, those skilled in the art will appreciate that the support member 108 can be detachably attached to the strap 104 by other types of securing mechanisms, such as by one or more of a buckle, button, snap, etc. In an alternative embodiment, the support member 108 is fixedly attached to the strap 104, such as by sewing the strap 104 to the support member 108 or a removable covering associated with the support member 108.

Referring again to FIGS. 1-12, and in accordance with at least one embodiment of the present invention, the shaped body 116 includes a concave interior surface 200 and a convex exterior surface 204. The concave interior surface 200 of the shaped body 116 allows the support member 108 to fit around the front of the neck and the hyoid bone of the wearer. More particularly, the concave interior surface 200 is shaped for maintaining the support member 108 firmly against the hyoid bone (or Adam's apple) of the wearer. When properly positioned, the support member tilts the wearer's head backward, thereby causing the tongue to move forward to open the airway.

In at least one embodiment, the support member 108 is preformed to possess a concave interior surface 200. More particularly, it is not necessary to bend the support member 108 to form its concave interior surface 200. Similarly, in at least one embodiment, the support member 108 is preformed to posses a convex outer surface 204, such that it is not necessary to bend the support member 108 to from its convex outer surface 204. By having a preformed concave interior surface 200, the strap 104 does not have to be overly-tightened to shape the support member 108 to the user's neck.

The configuration of the breathing enhancer 100 also maintains the jaw in a normal closed position in order to decrease soft tissue vibration in the back of the throat during sleep. That is, the strap 104 holds the support member 108 in a proper position for holding the chin in place, without protruding the mandible forward so that the support member 108 maintains the jaw in a closed or substantially closed position while creating a normal bite. This particular characteristic is particularly advantageous for longer-term health of the wearer. As those skilled in the art will appreciate, by not protruding the mandible forward, the wearer does not tend to develop jaw discomfort and bite misalignments. In use, the breathing enhancer 100 reduces or eliminates snoring in the user and potentially can lead to corrected posture long term. In reducing or eliminating snoring in the user, the teeth are maintained in a normal closed bite (1-5 mm open) while the lips are almost touching, thereby substantially eliminating open mouth breathing.

In accordance with embodiments of the present invention, the shaped body 116 can include other features. For example, the upper surface 208 may include a depression for receiving or cradling the underside of the wearer's chin. Thus, the support member can be made from multiple shapes and cutouts in a variety of materials.

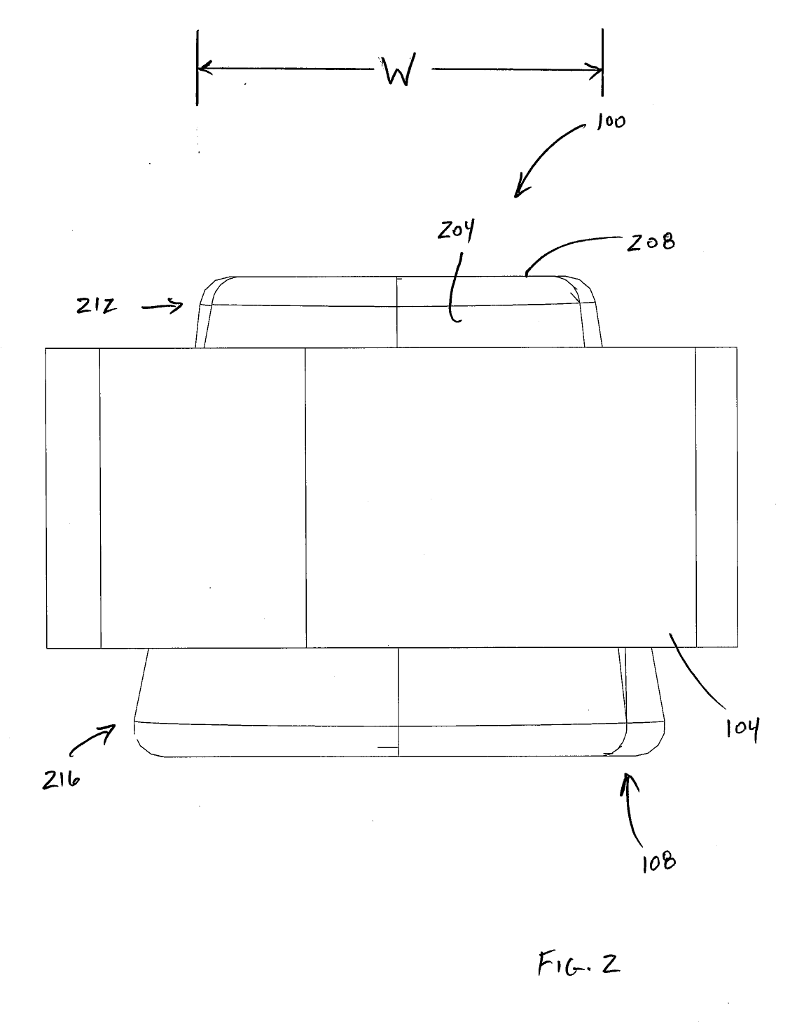

As those skilled in the art will appreciate, the support member 108 may be sized to fit most adults or sized to fit most children. Alternatively, the support member 108 may be capable of being custom-shaped to accommodate the specific characteristics of the individual wearer. By way of example and not limitation, for most adults the support member 108 comprises a width W of between about 3 to 8 inches wide, and more preferably, a width W of about 4-7 inches wide, and more preferably yet, a width W of about 5-6 inches wide. However, the width W of the support member 108 may vary. For example, as can be seen in FIG. 2, the width at the top 212 of the support member 108 is smaller than the width at the bottom 216 of the support member 108. In accordance with at least one embodiment of the present invention, the support member 108 does not wrap around the neck of the user. That is, the support member 108 has a width W that is no wider than the neck of the user and does not extend around the neck of the user.

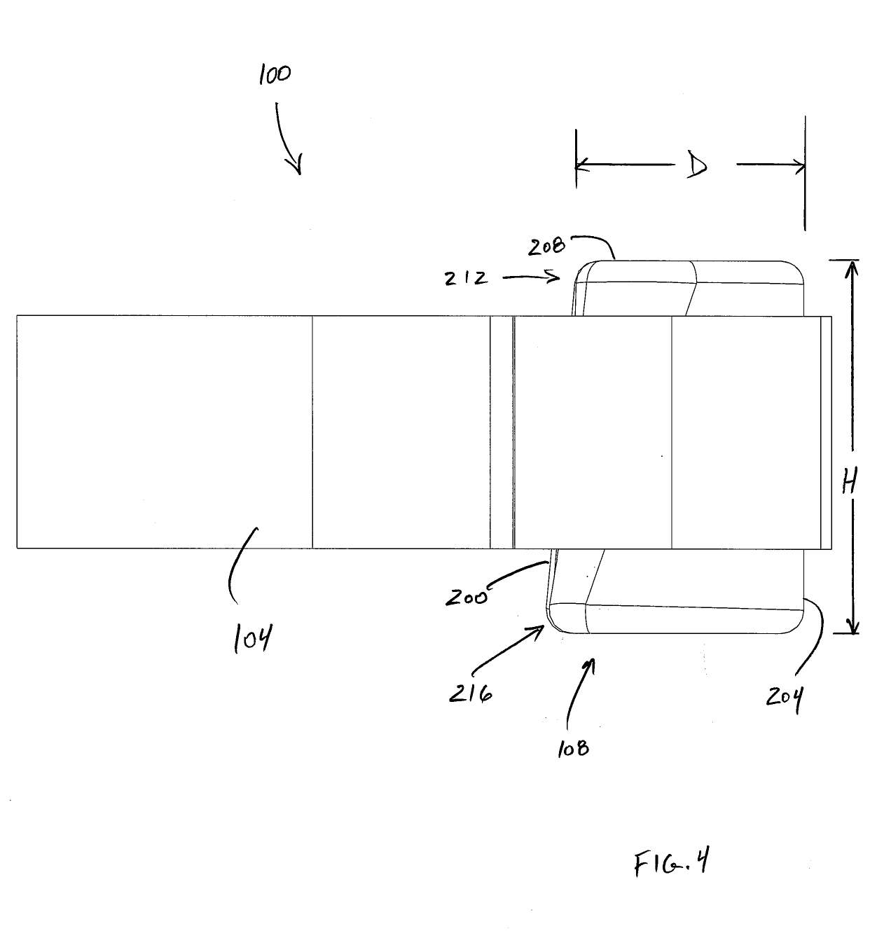

In addition, for most adults the support member 108 comprises a height H of approximately 1.5 to 5 inches tall, and more preferably, a height H of about 2 to 4 inches tall, and more preferably yet, a height H of about 3 inches tall. Also, for most adults the support member 108 comprises a depth D of approximately 1.5 to 5 inches, and more preferably, a depth D of about 2 to 4 inches, and more preferably yet, a depth D of about 3 inches. However, the depth D of the support member 108 may vary. For example, as can be seen in FIG. 4, the depth at the top 212 of the support member 108 is smaller than the depth at the bottom 216 of the support member 108.

The strap 104 has a length sufficient to allow the strap 104 to be adjustable with sufficient overlap to adequately engage the hook-and-loop fasteners. Accordingly, by way of example and not limitation, the strap 104 has a length of between about 16 to 24 inches, and more preferably, a length of about 18 to 22 inches, and more preferably yet, a length of about 20 inches long. The size of the hook-and-loop fastener may vary, but typically measures about 1.5 by 3 inches in size. In accordance with embodiments of the present invention, the strap 104 should be at least 2 to 3 inches wide at the back of the neck and produce a component of upward pressure, which will prevent the strap 104 from sliding down the neck.

The strap 104 and/or the support member 108 may also include a cloth covering, such as fleece or a soft, high thread-count cotton. In accordance with embodiments of the present invention, at least the exposed materials of the breathing enhancer 100 preferably are latex free, non-flaking, non-shedding materials that may be washed and cleaned.

Although the sizes given above are typical for a breathing enhancer 100 for adults, a children size can be easily provided in a proportionally smaller configuration. In accordance with embodiments of the present invention, the support member may comprise solid molded self-skinning urethanes at approximately a 15 lb density; cloth covered poly-foams in closed cell; foam with approximately a shore A of 15.25% compression ratio at approximately 4-6 psi; air-pillows with various baffle configurations, including user adjustable air bladders; and combinations of one or more of the foregoing.

It will be appreciated by those skilled in the art that the support member 108 may be held in place by a device other than a strap. By way of example and not limitation, a garment (not shown) may be provided that includes a pocket for receiving the support member 108 so that when worn, the garment maintains the support member 108 in a proper position.

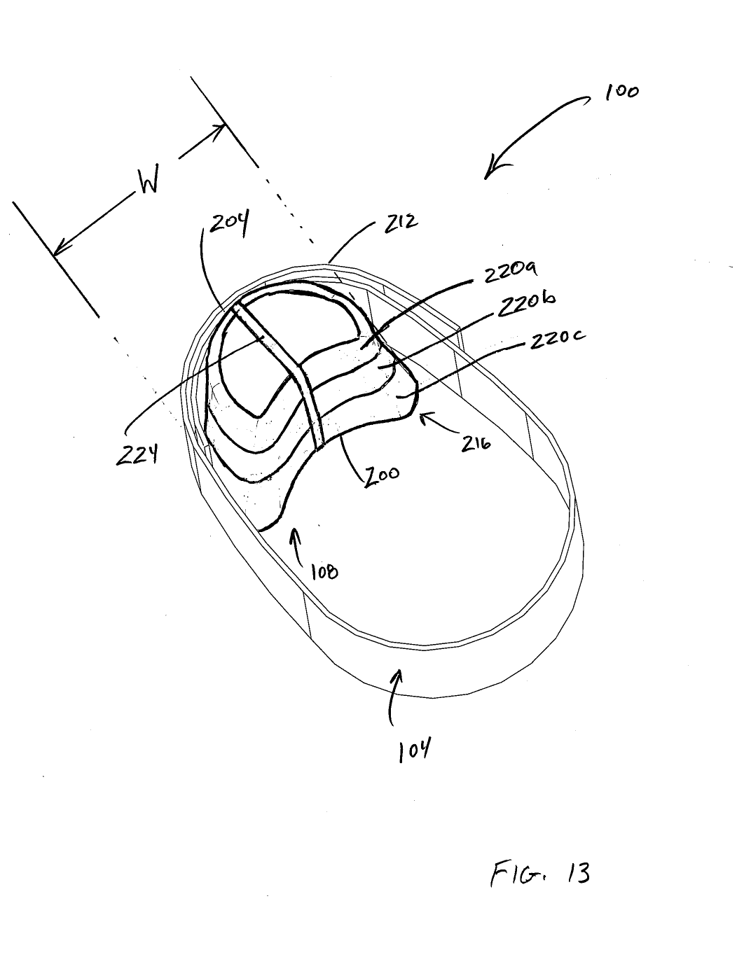

Embodiments of the present invention may include a support member 108 of a single-piece construction, or as seen in FIG. 13, the support member 108 may be a multi-piece construct have interconnectable portions or sections 220a, 220b, 220c, etc. For example, a first relatively small support member section 220a may be appropriate for a child. As the child grows, additional sections 220b and 220c can be added. The interconnecting mechanism between the sections 220a-220c may include a variety of devices, including by way of example and not limitation, a hook-and-loop fastener positioned between the sections (e.g., between 220a and 220b) and/or a wrap around strap 224 for securing the various sections 220a-220c together.

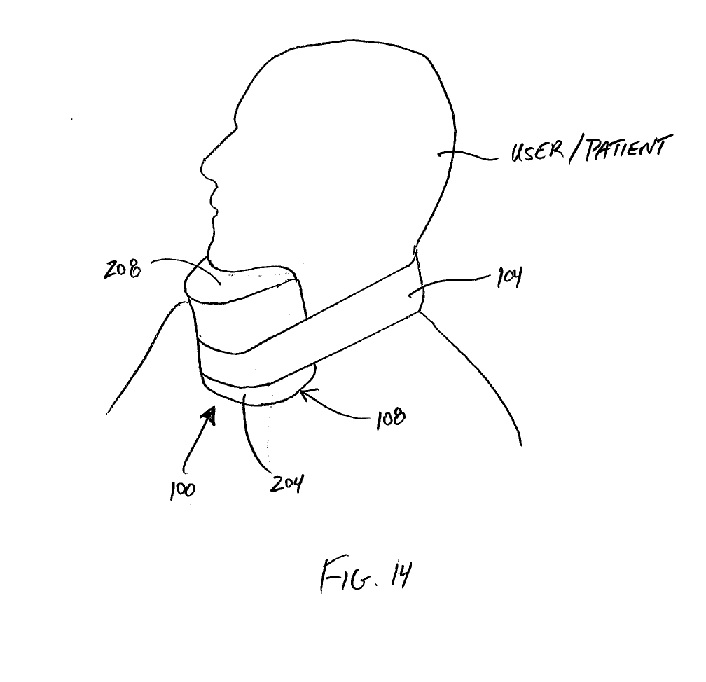

Referring now to FIG. 14, in use, the user places the support member 108 under his chin such that the concave surface 200 is located adjacent the front of the neck and in contact with the Hyoid bone or Adam's apple. The user then secures the strap around the neck to comfortably hold the support member 108 in place under the chin. Typically, a user will be able to sleep in any normal body position. Thus, the breathing enhancer 100 may be worn by a user when the user is lying on his back, or when sleeping in a bed in an alternate position, or when reclining in a chair. Regardless of the position of the user's body, the breathing enhancer 100 tilts the user's head backward and closes the lips together, including when the user is sleeping on his back.

In accordance with embodiments of the present invention, a method of treating snoring in a patient is provided, the method including providing a breathing enhancer 100 that includes a support member 108 and a way of holding the support member 108 below the user's chin. The user can secure and release the support member 108 to his person by, for example, securing a strap to hold the support member 108 in place under his chin. In at least one embodiment, the support member 108 is secured by a strap having a hook-and-loop type of fastener. The user can wear the breathing enhancer 100 when sleeping, whether the person sleeps lying down or reclined in a chair. The method of treating snoring may also comprise the step of continued use of the breathing enhancer to train the user as to proper sleeping posture. Additional steps may further comprise not using or intermittently using the breathing enhancer as part of a training program to reduce the need for using the breathing enhancer on a continuous basis. In accordance with embodiments of the present invention, proper use of the breathing enhancer can reduce snoring, and/or can lead to long-term snoring reduction without the need for any continued use of the device, and/or can lead to long-term snoring reduction with only intermittent use of the device.

In addition to the above described embodiments, and/or in accordance with at least one embodiment of the one or more present inventions, a device for enhancing breathing in a human user is provided, the user having a neck and a chin, and the user's neck having a front, a width, and a hyoid bone. The device includes a support member having an interior surface that is conformable to the user's neck front. The device is placed at the front of the neck of the user. In one or more embodiments, the support member laterally extends substantially no wider than approximately the width of the user's neck. Alternatively, all embodiments of the support members described herein may comprise a width that is permanently or at least temporarily wider than the user's neck. In at least one embodiment, the support member comprises an outer curved surface. The device further comprises a securing mechanism for holding the support member at the front of the user's neck.

In at least one embodiment, the securing mechanism is detachably attachable to the support member.

In at least one embodiment, the securing mechanism includes a strap having a portion for receiving the support strap therein. In at least one embodiment, the strap comprises a washable material.

In at least one embodiment, a plurality of securing mechanisms are provided, wherein the securing mechanisms are associated with more than one strap. In at least one embodiment, a first strap is oriented substantially around the user's neck, and a second strap is oriented in an oblique or perpendicular orientation relative to the first strap, such as around the top of the user's head.

In at least one embodiment, the support member comprises an adjustable mechanism for increasing the firmness of the support member. In at least one embodiment, the support member includes a bladder for receiving at least one of air and a liquid. In at least one embodiment, the size of the bladder is adjustable, such as by an air pump and/or valve. In at least one embodiment, the support member comprises foam. In at least one embodiment, the support member comprises memory foam that can be formed to accommodate the particular user's shape, such as the shape of the user's chin and/or neck.

In at least one embodiment, the support member includes a form-fitting shape, such as a kidney shape that accommodates the user's hyoid bone.

In at least one embodiment, the support member comprises an aperture and/or channel for accommodating a tracheal device.

In at least one embodiment, the support member includes a receptacle for an MP3 player. In at least one embodiment, the support member comprises and/or is operatively associated with one or more of a speaker, a timer, a vibrator, a light, a battery, an antenna, a radio transmitter, a radio receiver, and a telephone.

In at least one embodiment of the one or more present inventions, the breathing enhancer 100 is used in conjunction with an oral appliance that contacts one or more teeth of the user. By way of example and not limitation, a bite splint or other device that fits over a portion of one or more of the upper and/or lower teeth.

In at least one embodiment of the one or more present inventions, the breathing enhancer 100 is used by the user in conjunction with a device to treat sleep apnea, for example, a Continuous Positive Airway Pressure (CPAP) Mask.

In at least one embodiment of the one or more present inventions, the breathing enhancer 100 is used in conjunction with a passive device to enhance air flow through the user's nasal passages, such as nasal strip (e.g., BREATHE RIGHT® brand nasal strips).

In at least one embodiment, a strap for securing the support member comprises a way of adjusting its length, such as additional material that overlaps, extensions, elastic portions, insertions and/or add-on sections of material, multiple fasteners positioned at different locations along the strap, and combinations thereof.

In at least one embodiment, a back head support is provided in conjunction with the breathing enhancer 100, wherein an optional support for the back of the head is interconnected or interconnectable to the support member 108, such as by way of the strap 104. By way of example and not limitation, the back head support comprises a foam cushion that prevents the user's head from tilting too far backwards during sleep. In combination, the support member 108 and the back head support provided a range of motion within which the user's head is maintained.

While particular embodiments of the present invention have been described in some detail, it should be understood that other related embodiments are intended to be within the scope of the present invention. For example, other ways to functionally and structurally provide a device for supporting the head of the user in a position for enhancing breathing, whether such structures employ all or only some aspects of the present invention, and/or whether such structures are integrally made or form a connectable part of the apparatus or system described herein, such modifications, alternate uses and/or adaptations are within the scope of the present invention, including those that are well within the knowledge of those of ordinary skill in this art, regardless of whether such structures and/or uses are conventional or developed in the future.

The foregoing discussion of the invention has been presented for purposes of illustration and description. The foregoing is not intended to limit the invention to the form or forms disclosed herein. In the foregoing Detailed Description for example, various features of the invention are grouped together in one or more embodiments for the purpose of streamlining the disclosure. This method of disclosure is not to be interpreted as reflecting an intention that the claimed invention requires more features than are expressly recited in each claim. Rather, as the following claims reflect, inventive aspects lie in less than all features of a single foregoing disclosed embodiment. Thus, the following claims are hereby incorporated into this Detailed Description, with each claim standing on its own as a separate preferred embodiment of the invention.

While various embodiments of the present invention have been described in detail, it is apparent that modifications and adaptations of those embodiments will occur to those skilled in the art. However, it is to be expressly understood that such modifications and adaptations are within the spirit and scope of the present invention, as set forth in the following claims.

What is claimed is:

1. A device for enhancing breathing in a human user having a neck and a chin, the user's neck having a front, a width, and a hyoid bone, the device comprising:
a support member having a concave interior surface that is placed at the front of the neck of the user, wherein the support member laterally extends substantially no wider than about the width of the user's neck, and wherein the support member comprises an outer convex surface; and
a securing mechanism for holding the support member at the front of the user's neck.

2. The device of claim 1, wherein the securing mechanism comprises a strap.

3. The device of claim 1, wherein the support member comprises a top surface and a bottom surface, the top surface having a smaller surface area than the bottom surface.

4. The device of claim 1, wherein a bottom of the support member has a greater width than a top of the support member.

5. The device of claim 1, wherein a bottom of the support member has a greater depth than a top of the support member.

6. The device of claim 1, wherein the support member comprises a top surface for placement adjacent the chin of the user, and wherein the concave interior surface of the support member is configured for placement adjacent the hyoid bone of the user.

7. The device of claim 1, wherein the strap comprises a hook-and-loop fastener.

8. The device of claim 1, wherein the support member comprises a single-piece construction.

9. The device of claim 1, where in the support member comprises a multi-piece construction.

10. The device of claim 1, wherein the securing mechanism comprises a height less than a height of the support member.

11. A device for enhancing breathing during sleep in a human user having a neck and a head, the user's neck having a front, a width and a hyoid bone, the device comprising:
means for supporting the head of the user, the means for supporting comprising a preformed concave interior surface for placement adjacent the hyoid bone in the neck of the user; and
means for holding the means for supporting at the front of the user's neck.

12. The device of claim 11, wherein the means for supporting further comprises a convex outer surface.

13. The device of claim 11, wherein the means for supporting comprises a top width that is less than a bottom width.

14. The device of claim 11, wherein a top surface of the support member is substantially kidney-shaped when viewed in plan view.

15. The device of claim 11, wherein a width of the support member is no greater than the width of the user's neck.

16. A method of treating a human patient to reduce snoring, comprising:
positioning a support member adjacent to a neck of the patient, wherein the support member includes a concave interior surface for placement at a front of the patient's neck, wherein the support member laterally extends substantially no wider than about a width of the patient's neck, and wherein the support member comprises an outer convex surface; and
securing the support member to the patient's neck by a strap that can be released, the strap interconnected to the support member;
wherein the support member maintains a mouth of the patient in a substantially closed position, and wherein the support member tilts a head of the patient to maintain an open airway in the patient for aiding breathing and reducing snoring when the patient sleeps.

17. The method of claim 16, wherein the strap is detachably interconnected to the support member.

18. The method of claim 16, wherein said securing comprises engaging a hook-and-loop fastener associated with the strap.

19. The method of claim 16, wherein said positioning comprises placing a portion of the concave interior surface adjacent a hyoid bone in the patient's neck.

20. The method of claim 16, comprising providing at least the support member to the patient.
